(19) United States
(12) Patent Application Publication
(10) Pub. No.: US 2026/0223929 A1
(43) Pub. Date: Aug. 6, 2026

(54) EVAPORATOR FOR EXTRACTING AND FILTERING ACTIVE SUBSTANCES FROM BIOLOGICAL MATERIALS BY HEATING

(71) Applicant: BG BRAINGATE TECHNOLOGY GMBH, Köln (DE)

(72) Inventor: Fritz SCHMITT, Steinheim (LU)

(21) Appl. No.: 18/869,051

(22) PCT Filed: May 26, 2023
(86) PCT No.: PCT/EP2023/064277

§ 371 Date: Nov. 25, 2024

(30) Foreign Application Priority Data
May 27, 2022 (DE) 10 2022 113 395.8

(51) Int. Cl. A24F 40/46; A24B 15/167; A24D 3/04; A24D 3/06; A24D 3/16; A24D 3/17; A24F 7/00; A24F 40/20; A24F 40/42; A24F 40/48; A24F 40/60; A61M 11/04; A61M 15/06

(52) U.S. Cl. CPC A24F 40/46; A24B 15/167; A24D 3/041; A24D 3/061; A24D 3/166; A24D 3/17; A24F 7/00; A24F 40/20; A24F 40/42; A24F 40/48; A24F 40/60; A61M 11/042; A61M 15/06

(57) ABSTRACT

The invention relates to an evaporator for extracting and filtering active substances from biological materials by heating, characterized in that an active substance precursor in the form of a hollow cylinder, the outer walls of which are permeable to aerosols, is arranged in a heating chamber, wherein the active substance precursor is surrounded by a heating sleeve which is seated displaceably on the active substance precursor, wherein the length of the active substance precursor is a multiple of the width of the heating sleeve, preferably the width of the heating sleeve is approximately 5% to 25% of the length of the active substance precursor, so that a plurality, preferably approximately 4 to 15, partial regions of the active substance precursor can be heated individually, so that active substance provided by the active substance precursor can be smoked at least in regions and/or an aerosol which comprises active substances provided in regions by the active substance precursor can be generated.

### CROSS-REFERENCE TO RELATED APPLICATIONS

This application is a U.S. National Phase Application under 35 U.S.C. 371 of International Application No. PCT/EP2023/064277, filed on May 26, 2023, which claims priority to German Patent Application No. 10 2022 113 395.8, filed on May 27, 2022. The entire disclosures of the above applications are incorporated herein by reference.

### BACKGROUND

This section provides background information related to the present disclosure which is not necessarily prior art.

### TECHNICAL FIELD AND DISCUSSION

In medically therapeutic use, the inhaler/aspirator has taken up a fixed place, since not only active substances are used for inhalation, but also, depending on the indication, the prescription of whole cannabis flowers is used, which are decarboxylated in an evaporator in order to inhale the active substances extracted therefrom. In this case, the patient—especially non-smokers—is by no means to be expected to suffer the harmful burden of smoke during classical combustion.

With improved extraction (efficiency) using the inhaler at a low temperature level of up to a maximum of 200° C., far less damage to the lung system is detected than in the case of intake after combustion.

On the worldwide market, many different devices are offered which, using hot air, or using heating plates or glow wires, attempt to heat substances, herbs or liquids at a precisely defined temperature in such a way that the desired active substance is available for inhalation. Most of these devices operate according to the hearth plate principle. In this case, the herbs are distributed on a heated surface, in the hope that evaporation of the ingredients takes place in the process. This method functions only inadequately, since the substances which are in direct contact with the heated surface are heated to a considerably greater extent than layers which lie thereover, as a result of which reasonably uniform evaporation of the ingredients is ruled out. In the case of the other functional principle, the herbs are flowed through by air which has been heated to a specific temperature range and—assuming the necessary minimum temperature—evaporate their ingredients, which can then be inhaled after cooling of the air. In particular in the case of use in the medical sector, the devices which are on the market cannot guarantee device safety, as a result of which all the prerequisites for a medical device are absent. For example, the devices cannot be sterilized. This results in combustion residues on the heating plates or “grills”. The object of the invention is to provide a method and device according to the requirements of EU Regulation 2017/745 (Medical Device Regulation, MDR for short) which can heat active substances, in particular cannabis flowers, to 155°-200° C. in portions without an exothermic redox reaction. However, in the case of use of tobacco, a temperature of a maximum of 300° C. can also be provided.

The invention differs significantly in terms of construction from the inhalation devices which are on the market for herbal mixtures, cannabis and “heat not burn” devices for cigarettes.

With improved extraction (efficiency) using the inhaler at a low temperature level, far less damage to the lung system is detected than in the case of intake after combustion. In this case, the patient—especially non-smokers—is by no means to be expected to suffer the harmful burden of smoke during classical combustion.

The separation of the aerosols from the pollutants and condensate is an important component of the invention. None of the devices which are on the market guarantees pollutant-reduced inhalation. The novel invention guarantees a significantly lower pollutant burden. This is achieved by a plurality of systems filtering pollutants and collecting condensate.

The invention relates to “train inhalation evaporators” or to an evaporator of such train inhalation devices which permit intermittent, train-synchronous operation. Such an operating mode is present when the active substance portion is evaporated only during one train, while substantially no evaporation takes place at intervals between two trains. Train inhalation evaporators are inhalation apparatuses in which the vapor-air mixture or/and condensation aerosol formed can be supplied to the user in two steps, as in the case of a cigarette, namely first as a train into the oral cavity (first step) and after the evaporator has been set down in the form of a subsequent lung inhalation (second step). The train volume can vary individually here, between approximately 20 to 80 mL. In contrast thereto, in the case of “classical inhalation devices” the vapor-air mixture or/and condensation aerosol formed is supplied in a single step as direct lung inhalation. However, provision can also be made for the active substance aerosol produced to be able to be administered in only one inhalation operation.

In comparison with train evaporators, classical inhalation devices or evaporators have a significantly higher air throughput of approximately 100 to 750 mL/s compared with approximately 10 to 40 mL/s through the inhaler or evaporator. Evaporator. In the case of train evaporators, the comparatively low air throughput leads to a number of problems in conjunction with a high proportion of active substances or/and cannabis active substances. Precisely this problem is solved in the case of the novel invention in that a mixing ratio between active substance and air volume always remains the same, because only heated and thus active substance is produced when inhaled, and only by more train and/or inhalation can more active substance also be formed and mixed. Overdosing is ruled out in the case of the evaporator according to the invention.

Within the scope of the present patent application, an active substance, for example cannabis, as active substance precursor for CBD and/or THC is considered to be any active substance-containing solution whose mass proportion of or in the aerosol produced is only as high as permitted by the legal provisions.

An active substance proportion which is prescribed by law, for example in the case of use of cannabis as active substance precursor, has a number of advantageous effects. In the case of cannabis, the limits of the active substances are precisely defined, as early as in the rearing of plants.

Known evaporators are usually set up to heat cigarettes to be smoked and/or containers in which active substance can be accommodated as uniformly as possible. This can result in inhomogeneous heating and thus inhomogeneous mixture formation, for example from active substance and air, and/or inhomogeneous formation of active substance-containing aerosols. In particular if various active substances are provided which, for example, have different evaporation temperatures and/or temperatures at which active substance is or can be evaporated and/or extracted, or different amounts, in known evaporators the mixture formation and/or aerosol formation cannot be completely satisfactory.

### SUMMARY

This section provides a general summary of the disclosure and is not a comprehensive disclosure of its full scope or all of its features.

It is one aspect of the present invention to overcome these disadvantages.

The invention relates to an evaporator for extracting and filtering active substances from biological materials by heating, wherein an active substance precursor in the form of a hollow cylinder, the outer walls of which are permeable to aerosols, is arranged in a heating chamber, wherein the active substance precursor is surrounded by a heating sleeve which is seated displaceably on the active substance precursor, wherein the length of the active substance precursor is a multiple of the width of the heating sleeve, so that a plurality of partial regions of the active substance precursor can be heated individually, so that active substance provided by the active substance precursor can be smoked at least in regions and/or an aerosol which comprises active substance provided in regions by the active substance precursor can be generated.

The evaporator can have the heating chamber. The evaporator can have the active substance precursor. The evaporator can have the heating sleeve.

It can be provided that two different active substances can be provided in at least two regions of the active substance precursor, wherein the different active substances can differ, for example, in their composition and/or amount.

For example, smoking a cigarette with a significantly reduced pollutant absorption can be simulated and/or simulated with the evaporator. In principle, all active substances are conceivable which, for example, can be evaporated and/or extracted during a smoking process and/or by heating and/or heating, for example cannabis.

It can be provided that the width of the heating sleeve can be approximately 5% to 25% of the length of the active substance precursor. In some embodiments, approximately 4 to 15 partial regions of the active substance precursor can be heated individually.

The evaporator can have a mouthpiece. The mouthpiece can be configured to discharge and/or release an aerosol or the aerosol from the evaporator. The mouthpiece can be configured to discharge and/or release air from the evaporator. A user can inhale, inhale and/or receive the aerosol via the mouthpiece, for example.

The heating chamber can be formed double-walled with an inner wall and an outer wall. A cavity can be formed between the inner wall and the outer wall. It can be provided that the heating chamber can be a double-walled cylinder. A or the inner diameter of the heating chamber and/or heating chamber of the double-walled cylinder can be at least twice a diameter of the active substance precursor.

A vacuum can exist and/or be present in the cavity formed by the inner wall and the outer wall of the heating chamber. Alternatively, an active substance and/or coolant can be located in the cavity.

One or the inner wall of the heating chamber can have a nanocoating.

The evaporator can have a filter on a region of the heating chamber facing the mouthpiece. In some embodiments, the filter can comprise or consist of one or more of activated carbon, sponge and/or wet filter, statically charged metal wool, meltblown nonwoven or combinations thereof. The filter can be or have a molecular sieve.

The filter can have a plurality of segments and/or consist of a plurality of segments. In some embodiments, different pollutants can thus be filtered. It can be provided that each segment is configured and/or used for filtering a specific or specific harmful substance.

The evaporator can have a superabsorber on a region facing the mouthpiece, which superabsorber can be configured to collect condensate. Alternatively or additionally, the filter can comprise a superabsorber or the superabsorber.

The evaporator can have a line which can be configured to conduct outside air from an air inlet opening of the evaporator into the mouthpiece of the evaporator and/or into the heating chamber. It can be provided that the line is arranged in the heating chamber and/or can run at least partially in or through the heating chamber. It can be provided that the line is arranged in one or the cavity formed by the inner wall and the outer wall of the heating chamber, and/or can run at least partially in or through the cavity. The line can fluidically connect the air inlet opening to the heating chamber. Alternatively or additionally, the line can fluidically connect the air inlet opening to the mouthpiece.

An outer wall of the evaporator can be coated with thermochromatic lacquer. The thermochromatic lacquer can be configured to change its colour as a function of temperature. A user can thus identify at which position the heating sleeve is located, since there is a higher temperature locally in the heating chamber, for example, and the thermochromatic lacquer can accordingly have a different colour locally.

The evaporator can have a housing which can consist of a magnetic wire grid or can have a magnetic wire grid. The magnetic wire grid can be coated extensively with plastic. The plastic can, when current flows through wires of the wire grid, insulate the current-conducting wires from one another.

The evaporator can have at least one switching element arranged on or at a surface of the housing. The switching element can be inductively coupled to at least one wire and/or the magnetic wire grid. For example, it can thereby be made possible that a magnetic switching element can be placed on the surface at any desired point. The magnetic switching element can, for example, produce switching operations via induction. A switching operation can, for example, be or have a control command for a control of the evaporator. A switching operation can, for example, comprise an instruction that a smoking process is initiated, and/or the heating sleeve can be activated and/or controlled. A switching operation can comprise an initiation of a smoking process, and/or an activation of the heating sleeve.

The active substance precursor can form a receptacle. The receptacle can be a cylindrical receptacle. One or more active substances can, for example, be accommodated in the receptacle. The active substance precursor can be dimensioned such that it can have the at least one length of 8 cm, preferably at least 8.4 cm, and a diameter of at least 0.6 cm, preferably at least 0.8 cm. It can be provided that the active substance precursor can be dimensioned such that it completely accommodates a cigarette. It can be provided that the active substance precursor can be dimensioned such that a cigarette can be accommodated completely in the active substance precursor. The cigarette can be a commercially available cigarette, and/or have dimensions of a commercially available cigarette. The cigarette can contain one or more active substances.

In some embodiments, alternatively or additionally, the active substance precursor can be or comprise a cigarette.

It can be provided that the evaporator can be configured for smoking a cigarette accommodated in the active substance precursor, and/or can be used and/or used for smoking a cigarette accommodated in the active substance precursor.

The active substance precursor and/or the receptacle can have a cylindrical wire basket. The wire basket can be divided into a plurality of zones. It can be provided that the wire basket can be divided into at least five zones. It can be provided that the zones have no electrical contact with one another and/or can be electrically insulated from one another.

The heating sleeve can be movable. The heating sleeve can at least partially enclose the active substance precursor. The heating sleeve can have sliding contacts, which can be set up to supply the individual zones of the wire basket with current and/or to heat them. The sliding contacts can be set up to heat active substances accommodated and/or provided in a zone. The sliding contacts can be set up to form an aerosol. The sliding contacts can heat the active substance precursor in a region and/or a zone. By displacing the heating sleeve, corresponding regions can in each case be heated with the sliding contacts, e.g. one after the other.

The evaporator can have a sliding element, which can be arranged outside the heating chamber. The sliding element can be coupled to the heating sleeve, so that a movement of the sliding element can be transferred to the heating sleeve and/or vice versa. By displacing the sliding element, the heating sleeve can for example be displaced.

The sliding element can be elastically connected to the heating sleeve. The elastic connection can have a silicone tube, and/or the sliding element can be elastically connected to the heating sleeve by the silicone tube. The elastic connection can be or have an electrically conductive material. The sliding element can be elastically connected to the heating sleeve by the electrically conductive material.

The sliding element can have an active substance chamber, in which active substance can be received. Active substance can be accommodated and/or provided in the active substance chamber. It can be provided that the silicone tube can be configured to transport active substance from the active substance chamber into the heating chamber and/or the heating sleeve.

The active substance chamber can be reduced in size by pressure with the aid of a membrane, so that active substance can be fed into the tube. It can be provided that, by compressing the active substance chamber and/or the membrane, active substance can be fed into the silicone tube, the heating sleeve and/or the heating chamber, and/or can be fed, transported and/or conveyed into the silicone tube, the heating sleeve and/or the heating chamber.

The evaporator can have a deflection roller which can be configured to transport active substance into the heating chamber and/or the heating sleeve. The deflection roller can have convex bulges, for example. Alternatively or additionally, the deflection roller can be configured as a peristaltic pump. The deflection roller can be or have a peristaltic pump.

The heating sleeve can have a housing chamber wall which can have a filter material. It can be provided that air and/or aerosol formed in the heating sleeve can be diffused, and/or can diffuse, by the film material. It can be provided that aerosol which can be formed in the region of the active substance carrier enclosed by the heating sleeve can be diffused, and/or can diffuse, by the film material.

The housing chamber wall and/or the filter material can have pores which can be configured to change their size as a function of temperature.

One or the housing of the heating sleeve can consist of a material or have a material which can filter or bind harmful substances. The material can comprise or consist of one or more zeolites, preferably inactivated zeolites with a grain size of 0.1 to 2 mm, marble, activated carbon, clay, ash and/or a mixture thereof.

Zeolites can have, for example, the ability to exchange and bind surface cations on or with negatively charged platelets. A zeolite can also be or be referred to as “molecular sieve”, and/or be, have or correspond to a molecular sieve, since zeolites can filter a wide variety of cations.

The heating sleeve can have a seal. The seal can be or have a sealing lip. The seal can seal the heating sleeve. The seal can be configured such that, when the heating sleeve is moved, it compresses and/or reduces a region of the heating chamber. The seal can be configured such that, when the heating sleeve is moved, it compresses and/or reduces a region of the heating chamber arranged between seal and mouthpiece. The region which is or can be reduced and/or compressed by the seal can be arranged behind the seal in the direction of movement of the heating sleeve. The region can be arranged between mouthpiece and seal.

The heating sleeve can have a or the seal, preferably a or the sealing lip, wherein the seal can be configured such that, when the heating sleeve is moved, it conveys air, active substance and/or aerosol. In some embodiments, the seal can be configured such that, when the heating sleeve is moved, it conveys air, active substance and/or aerosol through a or the porous housing of the heating sleeve.

It can be provided that, during smoking, smoking and/or during or after the regional generation of the aerosol, the heating sleeve can migrate in the direction of the mouthpiece. It can be provided that the heating sleeve can be or can be moved in the heating chamber along a direction of extent of the active substance precursor.

The heating sleeve can have a heating sleeve. The heating sleeve can be or can be heated. The heating sleeve can be configured to release heat. The heating sleeve can be configured to heat and/or to heat the active substance carrier at least regionally.

The heating sleeve can have a memory alloy or consist of a memory alloy. The heating sleeve and/or the memory alloy can be configured to expand on heating and to contract on cooling.

The heating sleeve can have a front sealing disk and a rear sealing disk, between which the heating sleeve can be arranged and which can be connected to the heating sleeve. The front and/or the rear sealing disk can be configured to change its diameter.

The front sealing disk can be arranged closer to the mouthpiece than the rear sealing disk. The rear sealing disk can be arranged further away from the mouthpiece than the front sealing disk.

The front sealing disk and/or the rear sealing disk can each have a first part and a second part. It can be provided that the first part and the second part can be substantially semi-circular and/or semi-annular. The respective first part can be connected to the respective second part via a spring, so that the diameter of the respective sealing disk can be changeable, for example when the length of the spring changes and/or when the spring can expand or contract. It can be provided that the spring comprises a memory metal or can consist of such a metal. The memory metal and/or the spring can expand or contract depending on temperature. The length of the spring and/or of the memory metal can depend on the temperature thereof.

The front sealing disk and/or the rear sealing disk can be bendable. The front sealing disk and/or the rear sealing disk can be bendable in or by an angle of up to 130°. The diameter of the respective sealing disk can be changeable by bending between an unbent position and a bent position. It can be provided that the front sealing disk and the rear sealing disk can be configured to be bendable depending on temperature.

The front sealing disk and/or the rear sealing disk can be clamped to and/or contacts a or the inner wall of the heating chamber when the respective sealing disk is unbent. The respective sealing disk can be displaceable and/or movable in the heating chamber when the respective sealing disk is bent.

It can be provided that in the event of a temperature increase, the angle at which one or both sealing disks are bent can be almost completely reduced.

In some embodiments, the heating sleeve can thus be or can be moved. This can be achieved by the rear disk having an original shape without bend. The rear disk can clamp, clamp and/or lock on or with the inner wall of the heating chamber. The front disk can now be activated, for example by heating, and change its diameter, in that it is or can be transferred into the bent position, and/or in that it is or is bent by one or the predefined angle. The front disk can thus be movable, for example substantially in a direction along the active substance precursor. The heating sleeve can now be or can be activated, for example by heating, and expand substantially horizontally and/or in a direction along the active substance precursor. In this case, the front disk can migrate in the direction of the mouthpiece. After the heating process, the front disk can cool and clamp and/or lock on or with the inner wall of the heating chamber. The rear disk can now be activated, for example by heating. The rear disk can thus reduce its diameter and follow the cooling and contracting heating sleeve. Other possibilities of activating the disks, and/or one or the change in diameter of the disks, are also conceivable. The activation does not have to be limited to thermal activation. For example, one or both of the disks can be configured to change their respective diameter mechanically and/or by mechanical activation.

The evaporator can be configured to generate aerosol in the heating chamber by mixing active substance with air. The air admixed and/or to be admixed can be or comprise outside air.

The evaporator can be configured to generate aerosol by mixing active substance with air in the mouthpiece. The air admixed and/or to be admixed can be or comprise outside air.

The mouthpiece can comprise a flavouring substance and/or fragrance. The flavouring substance and/or fragrance can be encapsulated and/or encapsulated. Alternatively or additionally, the mouthpiece can have a porous material, through which flavouring substance and/or fragrance can pass.

The flavouring substance and/or fragrance can be encapsulated in a cellulose polymer. The flavouring substance and/or fragrance can be encapsulated in a cellulose polymer of wood pulp.

The mouthpiece can have a reservoir for the flavouring substance and/or fragrance. The mouthpiece can be configured to add and/or admix the flavouring substance and/or fragrance to a or the aerosol which is or will be conveyed through the mouthpiece. The mouthpiece can be configured to add and/or admix the flavouring substance and/or fragrance to a or the air which is or will be conveyed through the mouthpiece.

The mouthpiece can function as a flavour carrier and/or flavour carrier, e.g. in that the mouthpiece can store liquid or solid flavouring substances and/or flavouring substances in the reservoir, and/or the mouthpiece has a flavouring substance and/or flavouring substance or consists thereof.

A cellulose polymer of wood pulp is used for the encapsulating moulding compound. A coating with flavouring substances together with smell considerably increases the sensation of inhalation.

The evaporator can have a flap arranged in the mouthpiece, which flap can be configured to respond to suction. It can be provided that, when the flap responds to suction through the mouthpiece, a control element and/or the heating sleeve can be activated. An activation of the heating sleeve can comprise a heating of the heating sleeve and/or a heat release of the heating sleeve. Alternatively or additionally, it can be provided that, when the flap responds to suction through the mouthpiece, a or the aerosol is formed in the heating chamber and/or in the mouthpiece.

The mouthpiece can have a nozzle through which aerosol and/or air can be discharged from the mouthpiece.

The active substance precursor can be configured to accommodate and/or provide active substance. The active substance precursor can have a receptacle. Active substance can be accommodated and/or provided in the receptacle.

The receptacle can have one or more regions. Different active substances can in each case be accommodated in the receptacle in at least two different regions of the active substance precursor.

The active substance can be accommodated in an active substance provision unit. The active substance provision unit can be accommodated in the receptacle. The active substance provision unit can be or comprise a cigarette, and/or have a cigarette shape. The active substance provision unit can comprise a tank and/or a container. The active substance provision unit can be or comprise a capsule and/or the encapsulation. The active substance provision unit can be spherical.

The active substance precursor can have a filter material and/or consist of a filter material. The filter material can be configured to bind harmful substances arising during a heating process. In some embodiments, the filter material can comprise zeolite, and/or can comprise or consist of one or more zeolites.

The active substance can be encapsulated. The active substance can be encapsulated in spheres. A shell of the respective capsules and/or spheres can be configured to bind and/or filter harmful substances.

The active substance can be solid, liquid and/or gaseous. It can be provided that the active substance precursor can be configured to accommodate active substance in the solid, liquid and/or gaseous state.

The active substance precursor can be configured to bind active substances, and release them upon further activation, preferably upon heating. In some embodiments, the active substance precursor can be configured to bind active substances, which arise during the decarboxylation of cannabis, and release them upon further activation, preferably upon heating.

The evaporator can be configured to extract active substances, in particular CBD and/or THC, from plant material. The evaporator can be configured to extract active substances, in particular CBD and/or THC, from plant material. The evaporator can be configured to store active substances, in particular CBD and/or THC, in a medium. The plant material can comprise a cannabis plant or parts thereof.

The medium and/or the active substance can comprise ground cannabis flowers, and/or a mixture of ground cannabis flowers and flour, water, butter, baking powder, and/or sugar.

The evaporator can have a coupling which can be configured to dock an additional device on the evaporator and/or to connect it to the evaporator. The additional device can be or have, for example, a tank, preferably a tank for storing or providing gaseous substances, a vacuum pump, and/or a wet filter. The additional device can comprise a battery and/or an accumulator in order to provide, for example, electrical energy, in particular for the heating sleeve and/or the heating sleeve.

The evaporator can have a liquid reservoir which can be arranged on or in the mouthpiece. The liquid reservoir can be configured as a wet filter. The liquid reservoir can be arranged in such a way that generated aerosol and/or air can be conducted through the liquid reservoir. In some embodiments, it can be provided that the liquid reservoir can be arranged in such a way that generated aerosol and/or air must be conducted through the liquid reservoir before it can leave the evaporator.

The liquid reservoir and/or the wet filter can be configured to clean generated aerosol and/or to treat it with taste and/or smell. In some embodiments, the liquid reservoir can be or have a or the reservoir for flavouring substances and/or fragrances.

The heating sleeve can be configured to heat the active substance carrier to a temperature of up to 300° C., preferably to up to 180° C. The heating sleeve can be configured to heat the active substance carrier to a temperature of 300° C. The heating sleeve can be configured to heat the active substance carrier to a temperature of 220° C. The heating sleeve can be configured to heat the active substance carrier to a temperature of 180° C.

The evaporator can be configured to roast a cigarette, for example a commercially available cigarette, according to the “heat not burn” principle and/or to heat it to a temperature of up to a maximum of 300° C., without harmful substances being able to be released by burning in the process. As in the case of a normal smoking process, the cigarette can be heated in stages from the front to the rear, for example in stages of 3-5 mm, by means of the movable heating sleeve.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

The invention will be explained further by way of example with reference to the figures. In the figures:

FIG. 1 shows an exemplary embodiment of an evaporator according to the invention;

FIG. 2 shows another exemplary embodiment of an evaporator according to the invention;

FIG. 3 shows exemplary heating sleeves of some exemplary embodiments of evaporators according to the invention;

FIG. 4 shows a further exemplary embodiment of an evaporator according to the invention; and

FIG. 5 shows details of some exemplary embodiments of evaporators according to the invention.

### DETAILED DESCRIPTION

Example embodiments will now be described more fully with reference to the accompanying drawings.

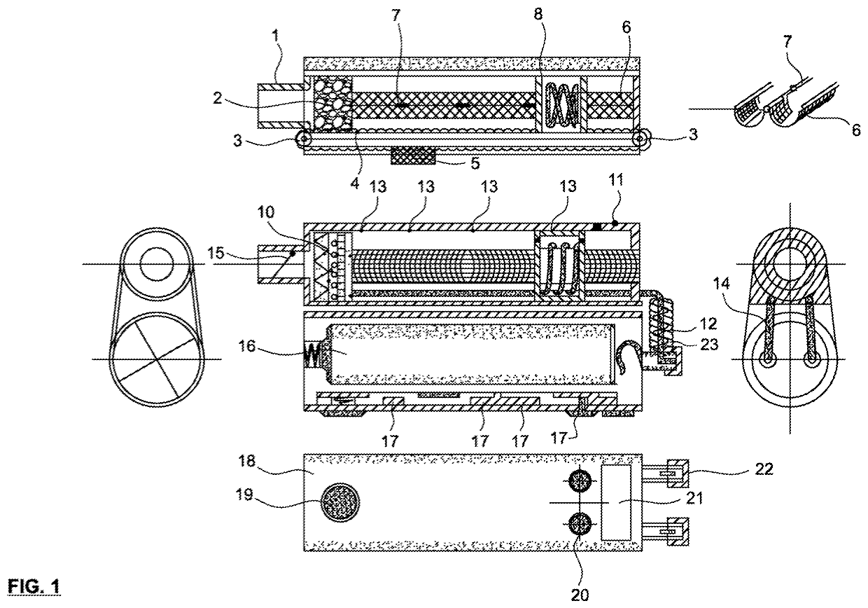

One, a plurality or all of the exemplary embodiments of evaporators according to the invention shown by way of example in one of FIGS. 1, 2, 3, 4 and 5 can have at least one, a plurality or all of the features and/or advantages of the exemplary embodiments shown by way of example in the respective other figures.

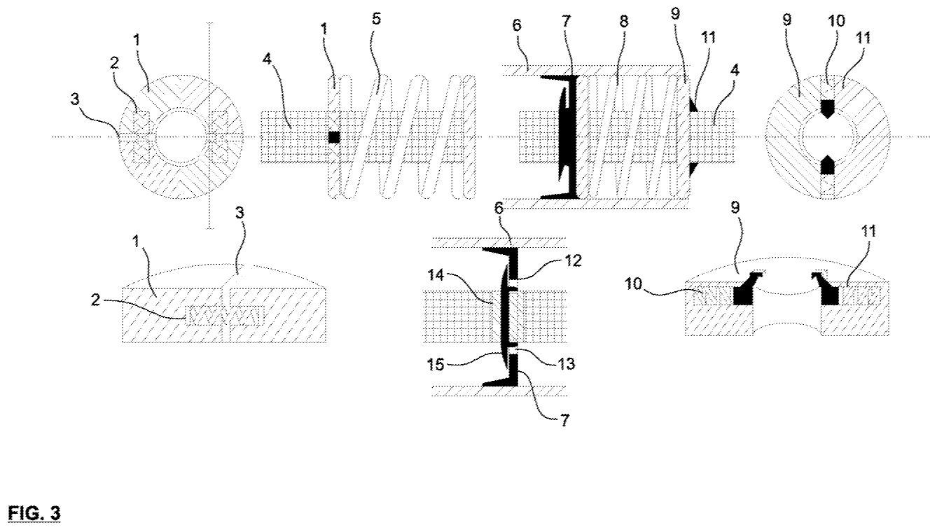

FIGS. 1, 2, 4 and 5 each show exemplary embodiments of evaporators according to the invention. FIG. 3 shows exemplary heating sleeves of some exemplary embodiments.

The evaporator has a heating chamber and an active substance precursor. The active substance precursor can be arranged in the heating chamber. The active substance precursor has the shape of a hollow cylinder and/or is cylindrical. The evaporator has a heating sleeve. The heating sleeve surrounds the active substance precursor and is arranged so as to be movable relative thereto. The heating sleeve can be arranged in the heating chamber. The active substance precursor has one or more regions. The heating sleeve can cover a region. When the heating sleeve is or will be displaced and/or moved along the active substance precursor, the heating sleeve can cover a different region.

An active substance can be accommodated and/or arranged in the active substance precursor. The active substance can, for example, be accommodated in an active substance provision unit and/or provided by an active substance provision unit. The active substance provision unit can, for example, be a cigarette which can contain the active substance. Alternatively or additionally, the active substance provision unit can be or comprise an encapsulation, in which active substance can be accommodated and/or encapsulated. In some embodiments, the active substance provision unit can be or comprise a container, a tank, or the like, in which active substance can be accommodated and/or arranged. The active substance provision unit can be accommodated in the active substance precursor. For example, the active substance precursor can have a receptacle, in which active substance and/or the active substance provision unit can be accommodated and/or arranged.

In some embodiments, the length of the active substance precursor can be a multiple and/or multiple of a length of the heating sleeve. The multiple and/or multiple does not have to be an integer multiple and/or multiple.

The heating sleeve can heat and/or heat the active substance precursor and/or active substance accommodated in the active substance precursor and/or an active substance provision unit accommodated in the active substance precursor. Upon heating of the active substance precursor, the active substance and/or the active substance provision unit, active substance can escape, evaporate and/or be extracted. The escaped, evaporated and/or extracted active substance can be mixed with air in order, for example, to form an active substance-containing aerosol.

In some embodiments, the heating sleeve can be or can be displaced manually and/or by a user. For example, a slider and/or a sliding element can be provided for this purpose, which can be mechanically coupled to the heating sleeve. By displacing the slider, the heating sleeve can be or can be displaced accordingly, in particular along the active substance precursor. Successively different regions of the active substance precursor can thus be heated, and active substance can accordingly escape, evaporate and/or be extracted and/or an aerosol corresponding to regions can be formed.

In some other embodiments, alternatively or additionally, it can be provided that the heating sleeve can be or can be moved, for example, via a drive. In some embodiments, the drive and/or the movement of the heating sleeve can be controlled and/or regulated. It can be provided that the movement of the heating sleeve can depend on the type and/or amount of the respective active substance.

In some embodiments, alternatively or additionally, it can be provided that the heating sleeve can move independently and/or automatically along the active substance precursor.

In some embodiments, the active substance accommodated and/or provided in the active substance precursor and/or the active substance provision unit can thus be or can be successively evaporated, extracted and/or smoked in the heating chamber.

The evaporator can have a mouthpiece. The mouthpiece can be or can be fluidically connected to the heating chamber. Aerosol formed and/or generated, in particular active substance-containing aerosol, can escape through the mouthpiece and/or be supplied to a user. The aerosol formed and/or generated, in particular active substance-containing aerosol, can thus be or can be smoked by the user.

It can be provided that the evaporator can have a filter between mouthpiece and heating chamber, and/or a filter can be arranged in the heating chamber. The filter can serve to filter pollutants out of the aerosol. It can be provided that the aerosol must pass through the filter in order to enter the mouthpiece.

The heating sleeve can have a heating sleeve. The heating sleeve can be or correspond to a heating spiral. The heating sleeve and/or the heating sleeve can be configured to convert electrical energy into heat. For example, the heating sleeve and/or heating sleeve can have and/or consist of a suitable metal and/or a suitable metal alloy. The heating sleeve and/or heating sleeve can have an electrical resistance which is selected such that, when electrical current flows through the heating sleeve and/or heating sleeve, the heating sleeve and/or heating sleeve is heated and/or is heated.

It can be provided that the active substance precursor can extend through and/or into the heating sleeve. In some embodiments, the heating sleeve can be wound around the active substance precursor. It can be provided that the heating sleeve can be arranged and/or shaped such that it does not contact the active substance precursor. In some embodiments, the heating sleeve can have a front and a rear sealing disk. One or both sealing disks can be shaped such that the active substance precursor can extend through the respective sealing disk. It can be provided that the heating sleeve can be connected to the sealing disks, and/or can be fastened to the sealing disks. It can be provided that the sealing disks can be electrically insulated from the heating sleeve.

The evaporator can have an electrical energy store, for example a battery and/or an accumulator. The electrical energy store can be configured and/or serve to provide electrical energy, for example current. It can be provided that the electrical energy store can supply the heating sleeve and/or the heating sleeve with electrical energy, in particular current.

Further advantages and/or features are described below.

A condensate or the condensate can be collected in a superabsorber (FIG. 1. 10) provided for this purpose, which superabsorber can be part of a or the filter unit. The inner wall of the heating chamber (FIG. 1. 7) can be provided with a microscopic and nanoscopic architecture of the surface, which can minimize the adhesion of dirt particles. In the lower part of the inner surfaces of the heating chamber (FIG. 1. 7), strips can be attached to surfaces with superabsorber (FIG. 1. 10). After the inhalation operation, the heating chamber (FIG. 1. 7) can be opened once and the superabsorber (FIG. 1. 10) can be removed.

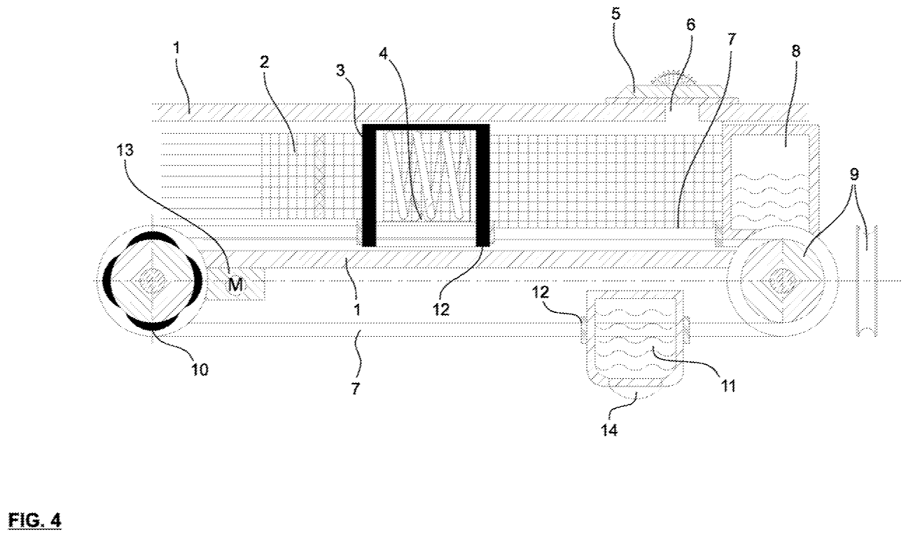

The invention and/or evaporators according to the invention can heat only a small part of active substance precursor (FIG. 3. 4) on each inhalation. This is achieved in that a hollow-cylindrical active substance precursor with the approximate dimensions of a cigarette can be arranged in a tubular housing (FIG. 1. 18), which can be so large in diameter that the active substance precursor (FIG. 3. 4), which can be attached centrally in the tube, can have a circumferential distance of at least 3 mm from the inner boundary of the housing tube (FIG. 1. 18). The active substance precursor can also be present in the form of a sphere. The heating chamber (FIG. 1. 7) can be double-walled. A vacuum can be provided between the inner and the outer wall. An embodiment can provide that other substances can also be located in the cavity, e.g. coolant, or substances which can diffuse into the heating chamber (FIG. 1. 7) and/or which can have an effect on harmful substances which can arise in the heating chamber (FIG. 1. 7) during the heating process. This effect on harmful substances can be similar to the influence of AdBlue in or on diesel exhaust gas. The active substance precursor can have a carrier. A special feature of the invention can be that the carrier can be surrounded circumferentially by a heating sleeve (FIG. 4. 3) in a zone which can correspond to 5%-10% of the length of the carrier. The heating sleeve (FIG. 4. 3) can draw its energy, or energy, from two contact rails. The contact rails can have contact surfaces in very specific regions in order to supply the heating element and/or the heating sleeve, and/or functional elements, with current. The heating element can be or comprise a heating unit. The heating sleeve can be or comprise the heating element and/or the heating unit. The heating unit can be a significant part of the invention because, as in the case of burning, e.g. a cigarette, it can heat in each breath an amount of the active substance precursor which can correspond to the length of the heating unit, e.g. a maximum of 5 mm. The heating unit can release another region, e.g. a or the front region of the heating chamber (FIG. 1. 7). The heating unit and/or the heating sleeve can migrate in the direction of the filter unit and/or can be movable. In this case, the heating space can increase in each breath and accumulated heat can become smaller, the further the heating unit can move away from the filter. For the movement of the heating unit, in some embodiments the temperature of the heating spiral can be used which can arise during activation. The heating unit can have or have at least one contact point, a closure disk (FIG. 1. 8) at the front, a heating spiral, a closure disk (FIG. 1. 8) at the rear, for power supply, which in some embodiments can also be present or guided in parallel on the contact rails. During the inhalation operation, contact 3 can be activated. Memory springs can expand and increase the diameter of the closure disk, which can clamp to the inner housing wall (FIG. 1. 18). At the same time and/or in this case, the contact 2 and 1 can be activated, and the coil can become approximately 200° hot and expand. The memory springs of the closure disk can be configured and/or programmed such that they can contract in the event of temperature changes. The closure disk 1 can migrate with the heating spiral in a prescribed direction, by 3-5 mm depending on the programming or device. As soon as the aspiration operation is ended and the heating spiral cools, the front closure disk can block by increasing the diameter. The heating spiral can contract and entrain the rear closure disk (FIG. 1. 8), which can expand again on cooling. The displacement of the heating unit can alternatively or additionally also take place mechanically from the outside with a slider.

Advantageously, the active substance precursor or the active substance carrier can be ejected after use and after removal of the mouthpiece with the aid of the heating element which is not in operation. In addition, the invention makes it possible that the inhalation operation can also be interrupted for a longer period of time.

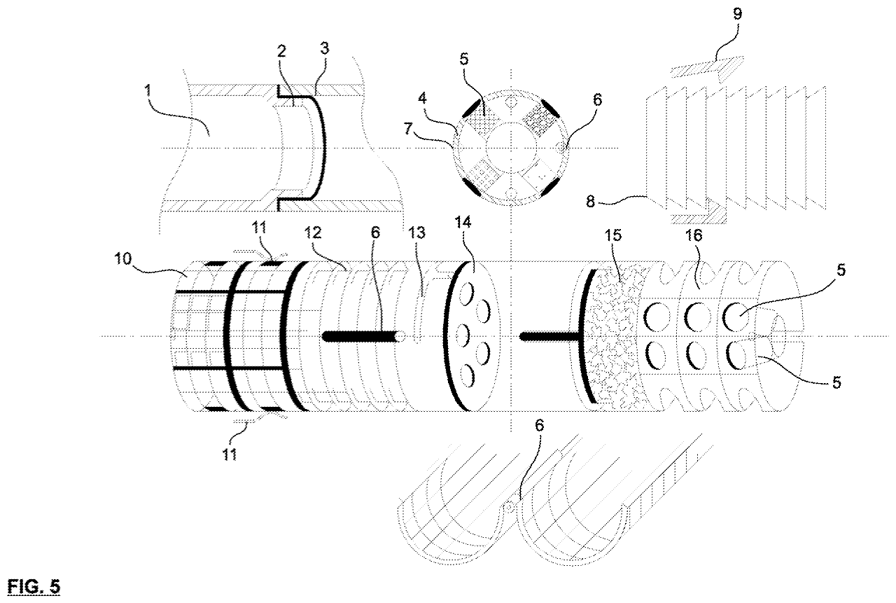

Embodiments of the invention comprise an active substance precursor, which can also be referred to as active substance precursor carrier (FIG. 5) or WV carrier.

The WV carrier can have the task of connecting active substance constituents, active substances and materials which can have a favorable effect on the properties of the aerosol generated, to one another, and/or of using supplied minimum energy in such a way that active substance constituents, active substances and materials are freed of harmful substances by heat and/or heat which are released at certain temperatures and by the material properties of the active substance precursor carrier. This can be achieved by a part of the active substance precursor carrier consisting of or being able to comprise zeolites. For example, ground flower material of the cannabis plant can be incorporated into pores of the active substance precursor carrier. It can be provided that the CBD or THC can or must diffuse through the zeolite material on heating, wherein harmful substances cannot break through this barrier.

The active substance precursor carrier can have a technically dependent shape which can correspond approximately to the dimensions of a cigarette. The active substance precursor carrier can consist of a plurality of chambers which, in some embodiments, contain different active substances and/or can be connected to one another. Silicone separating disks can, for example, separate each part and/or the chambers of the active substance precursor carrier from one another in a gas-tight manner. An electrical conductivity can be interrupted by insulating seals and/or silicone disks. Each chamber of the active substance precursor carrier can be activated independently of one another. Activating can mean heating a chamber and/or an active substance, active substance constituent or other material accommodated in a chamber. Activating can mean releasing and/or releasing an active substance accommodated in a chamber. Activating can mean generating or forming an aerosol, for example by mixing active substance with air, for example released and/or released active substance and air. The materials used can in principle be reusable or biodegradable.

The heating sleeve (FIG. 4. 3) can comprise or consist of various porous materials, for example zeolites, foamed metals or the like. The porous materials can make it possible that the material which is located in the active substance carrier and can generate aerosols on heating can be freed of harmful substances on diffusing through the chamber wall of the heating sleeve. In some embodiments, the active substance precursor carrier can consist of material or have a material which can function as a molecular sieve (FIG. 1. 2). It can be provided that the pore size can be matched accordingly to the harmful substances. In the case of some substances, a plurality of layers of the active substance precursor carrier wall can be necessary, and/or a multilayered active substance precursor carrier wall can be provided in order to achieve optimum filtering. It is conceivable that active substance and/or active substance constituents can be introduced into chambers of the active substance precursor carrier. The chambers and/or their walls can be selected from a material which can change its pore size depending on the temperature of the chamber and/or its walls. The active substance precursor carrier can form its own functional unit by virtue of its construction and supply of energy, and/or carry out chemical processes.

The invention relates to an evaporator for the formation of an active substance-containing aerosol vapor-air mixture or/and a liquid flavour carrier from condensation aerosol by evaporation by means of a heating sleeve (FIG. 4. 3) and an active substance precursor.

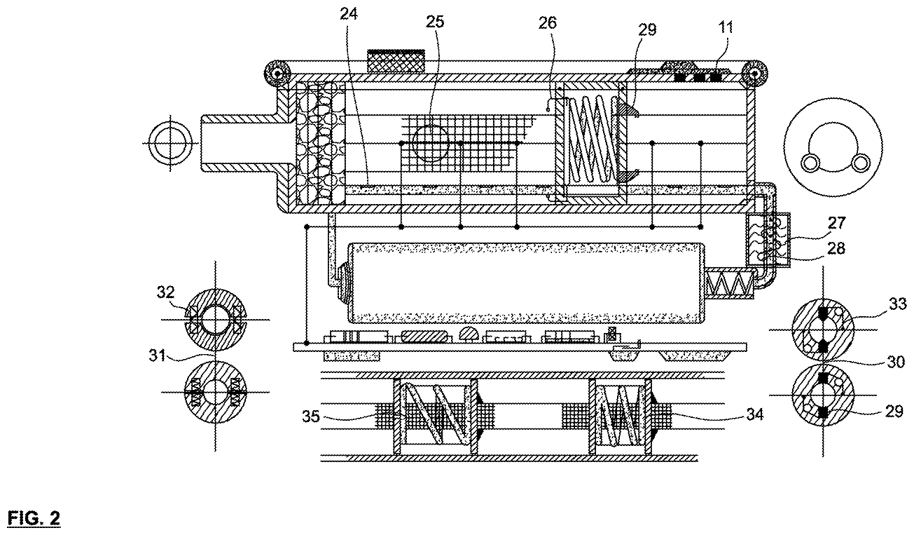

In some embodiments, the evaporator can comprise a double-walled housing (FIG. 1. 18). One or the active substance precursor carrier (FIG. 1. 6) can be arranged in the housing (FIG. 1. 18). The evaporator can have an air inlet opening (FIG. 2. 11) for the supply of air from the surroundings into the chamber or mouthpiece (FIG. 1. 1). The evaporator can have a or the heating sleeve (FIG. 4. 3) for evaporation of a specific amount of active substance in a or the heating chamber.

With a or the heating sleeve (FIG. 4. 3) arranged in a or the heating chamber, an aerosol and/or a vapor/air mixture can be generated in the heating chamber (FIG. 1. 7) (FIG. 4. 1). The aerosol and/or the vapor/air mixture can be generated or formed by mixing in the chamber or in the mouthpiece (FIG. 1. 1) with the air supplied through the air inlet opening (FIG. 2. 11). In some embodiments, a multi-stage pollutant separating device and/or a filter (FIG. 1. 10) can be provided in order to separate as far as possible a high pollutant ballast in the vapor-air mixture or condensation aerosol formed. The pollutant separating device and/or the filter can have (or consist of) a zeolite active substance carrier unit, e.g. a zeolite filter unit (FIG. 1. 10) communicating and/or fluidically connected to the heating chamber (FIG. 1. 7) (FIG. 1. 7). Alternatively or additionally, it can be provided that the pollutant separating device and/or the filter is or has a molecular sieve (FIG. 1. 2) through which vapor-air mixture or/and condensation aerosol formed can flow and can filter and/or bind it.

In the present patent application, the term “evaporator” can relate to medical and non-medical aspiration and inhalation apparatuses, and/or comprise such. Smoking articles and cigarette replacement articles, as are contained, for example, in European patent class A24F47/00B, can also be meant or comprised insofar as they are intended to supply the user with a vapor-air mixture or/and active substance-containing aerosol, e.g. active substance-containing condensation aerosol.

The term active substance (FIG. 1. 6) can relate to all substances which release active substances upon decarboxylation, change their state of aggregation upon decarboxylation, and/or which can be administered by inhalation.

It can be provided that the active substance can have a high proportion of propylene glycol or/and water. This can result in an improvement in the dosability of the active substance, a general improvement in the solubility for further ingredients in the active substance, or an improvement in various physical properties, such as, for example, a reduction in the viscosity of the active substance. The latter effect can, for example, come into play when promoting the active substance and/or when the active substance is or is to be atomized. Water can in addition prevent the hygroscopicity of the aerosol formed and thus prevent the oral cavity and the throat from drying out. Propylene glycol and water can together also have a germicidal action and thereby improve the hygiene of aerosol generation and aerosol administration.

In its own extensive series of tests, it was unexpectedly found that the enjoyment of condensation aerosols generated on the basis of such highly dilute active substances in or by inhalation devices and/or evaporators according to the invention with disadvantageous organoleptic effects can be associated, inter alia, with an irritating taste stimulus on the tip of the tongue and the lips. The cause for this can be associated with the large proportion of harmful substances which can arise during burning of biological material such as cannabis. Corresponding mechanisms of action are known, for example, from smoking.

In some embodiments of the invention, the proportion of harmful substances can be considerably reduced since active substances such as tobacco, cannabis or herbal mixtures can only be heated but not burned. Through the use of the new filter technology using zeolites, harmful substances can be filtered/bound in a targeted manner. Nevertheless, in some embodiments, it cannot be ruled out that residual moisture loaded with harmful substances can be released during heating of the biological active substances. The formation of condensate cannot be prevented in some embodiments because, for example, in the case of cannabis, a residual moisture of up to 20% can usually be bound in the plant material. However, this residual moisture can be useful during decarboxylation, for example during heating of cannabis material. The evaporation of the residual moisture can begin at a temperature of 100° C. During a first heating process, the aerosol can already have and/or transport a quantity of harmful substances. In this case, it can be conceivable that no inhalation is started. The first heating phase can substantially also be responsible for the movement of the heating sleeve (FIG. 4. 3). It can be provided that during the movement, no inhalation takes place and/or is not inhaled when the heating sleeve is or will be moved. It is advantageous in the case of cannabis that the desired active substances are only released from 150°. In the case of cigarette products, active substances, such as for example nicotine, can in some embodiments only be activated at 250°-300°.

The invention can overcome the above-described and/or previously indicated disadvantages. Embodiments of the invention can in particular be based on the object of configuring an inhalation device in such a way that one or more of the following properties are realized. In some embodiments, a hollow cylinder can serve as active substance precursor carrier (FIG. 1. 6). The walls of the hollow cylinder can be open-pored walls and/or have a lattice structure. The hollow cylinder can be enclosed by a heating sleeve (FIG. 4. 3). The heating sleeve can be displaceable on the hollow cylinder. The length of the heating sleeve (FIG. 4. 3) can in some embodiments be approximately 5%-10% of the length of the hollow cylinder. The length of the hollow cylinder can be approximately 70-100 mm. On one, more or each activation of the heating sleeve (FIG. 4. 3), the heating sleeve (FIG. 4. 3) can heat the active substance precursor and/or active substance. On and/or after one, more or each activation, the heating sleeve can be or can be moved along the hollow cylinder. For example, the heating sleeve can migrate by approximately 3-5 mm on the hollow cylinder, for example on and/or after one, more or each activation. It can thus be ensured that only a small part of the active substance precursor can be or can be heated. The temperature can be selected such that burning of the active substance and/or of the active substance precursor can be ruled out. The method can simulate and/or simulate smoking of a cigarette, but without generating harmful substances. Other materials can also be used and/or can be used which release active substances, in particular cannabis, during the smoking process.

Part of the invention can relate to the movement of the heating sleeve (FIG. 4. 3) using temperature. The movement can be possible, for example, through the use of memory spring elements. The memory spring elements can make possible the movement by expanding or compressing the spring elements. The heating sleeve can be self-migrating. A or the heating sleeve, in particular a or the self-migrating heating sleeve, (FIG. 4. 3) can be closed at the front and rear by displaceable cover disks (FIG. 2. 32, 33). The cover disks can be or have or correspond to sealing disks. The cover disks can be connected to the heating sleeve (FIG. 3. 8). It can be provided that the cover disks are not connected to the heating sleeve (FIG. 4. 3). The cover disks can be separated in diameter (FIG. 2. 33). The cover disks can have two parts. The parts can in each case be semicircular parts. The two parts can be displaceably connected to a tongue and groove mechanism, and/or to a spring. The two semicircular parts (FIG. 2. 32) can be connected to a train and/or compression spring made of memory alloys. The spring and/or the springs can be attached such that the cover disks can increase or reduce their diameter. The bore in the disks can correspond to the diameter of the hollow cylinder (FIG. 1. 6). The hollow cylinder can be guided through one or both cover disks and/or extend through one or both cover disks. The outer diameter of the cover disks can be selected such that, on activation of the memory spring, a disk, in particular the rear disk, can be supported on the inner wall of the heating chamber (FIG. 1. 7) (FIG. 4. 1) or on the hollow cylinder.

The front boundary disk, or the front cover disk and/or front sealing disk, can be immobile in a rest state and can be supported on the inner wall of the housing (FIG. 1. 18), and/or the inner wall of the heating chamber and/or of the hollow cylinder. The disk, or the front boundary disk, can contract under heat and/or heat which can emanate from and/or be released by the heating spiral made of memory alloy (FIG. 3. 5) in the first heating phase at a temperature of approximately 60°-120°. In this case, the front boundary disk of the heating sleeve (FIG. 3. 8), which can expand at the temperature reached, can yield and migrate forward, and/or into or against the mouthpiece. On decay of the temperature, the disk can expand and fix at its new position. The heating unit can contract on cooling, wherein the rear boundary disk can respond and can follow its movement forward (and/or into or against the mouthpiece). This can be made possible since the clamping (FIG. 2. 29) can be released or can only grip in one direction. It can be provided that, after cooling of the front disk, the rear disk can heat, and/or that, after locking of the front disk, the rear disk can be released. It can be provided that the rear disk can thus move and/or pull, and/or can move.

Alternatively or additionally, it can be provided that the heating sleeve (FIG. 4. 3) can be moved from the outside with a sliding device (FIG. 4. 7). This can be achieved by the heating sleeve (FIG. 4. 3) being able to be connected to a tube-like element. The tube-like element, for example a tube and/or a silicone tube, can be connected via at least one deflection roller (FIG. 4. 9). The deflection roller can have convex bulges (FIG. 4. 10) on the running surfaces in order to be able to exert a pumping action on the connecting tube. Material, for example active substance, can thus be transported into the heating sleeve (FIG. 4. 3) (FIG. 4. 3). The sliding element can have a hollow body/tank (FIG. 4. 12), which can be, have or correspond to an active substance chamber. It can be provided that active substance can be introduced and/or accommodated in the cavity (FIG. 4. 11) or the active substance chamber. An elastic membrane, which can be designed and/or formed, for example, as a push button (FIG. 4. 14), can make possible a transport of active substance into the heating chamber (FIG. 1. 7) or heating sleeve (FIG. 4. 3), in particular a transport of active substance out of the active substance chamber.

In some embodiments, the amount of aerosol to be filtered can be greatly reduced. This can be achieved by the aerosol, which is not in the heating chamber (FIG. 1. 7) but in the mouthpiece (FIG. 1. 1), being able to be mixed and/or generated with fresh air. It can be provided that active substance can be evaporated and/or at least partially transferred into a gaseous state in the heating chamber and/or the heating sleeve, which can be mixed with fresh air in the mouthpiece.

It can be provided here that the venturi effect can be utilized.

Alternatively or additionally, a or the aerosol can be or can be formed in the heating chamber and/or the heating sleeve. It can be provided that active substance and fresh air can be or can be mixed in the heating chamber and/or the heating sleeve. It can be provided that active substance can be evaporated and/or at least partially transferred into a gaseous state in the heating chamber and/or the heating sleeve, which can be mixed with fresh air in the heating chamber and/or the heating sleeve.

The fresh air can be or have ambient air and/or outside air, for example. The fresh air can be introduced and/or supplied through an or the air inlet opening.

The mouthpiece (FIG. 1. 1) can consist of a porous material or have such a material. It can be provided that flavouring substances and/or fragrances, e.g. complex fragrances, can be encapsulated, e.g. encapsulated in plastic parts. The flavouring substances and/or fragrances can be released over longer periods of time. A cellulose polymer, e.g. a cellulose polymer of wood pulp, can be used for the encapsulating moulding compound. A coating with flavouring substances can, e.g. together with smell, considerably increase the sensation of inhalation. The mouthpiece (FIG. 1. 1) can release flavouring substances and/or smell substances. In some embodiments, the mouthpiece (FIG. 1. 1) can consist of a material or have a material which can store fragrance and/or release flavouring substances. The mouthpiece (FIG. 1. 1) can also be formed as an exchangeable filter unit, which can be provided and/or formed rotatably and in an angled manner. The function can be designed as in the case of a bendable drinking straw. In some embodiments, the evaporator can activate and/or release flavouring substances, which can supply the tongue and the inner nasal space of a user with aerosols as flavour carriers.

In fact, usually only a small part of the sensations perceived as taste (in a broad sense) is based on irritations of the taste receptors. The often by far predominant part is frequently caused by volatile flavouring substances, which can irritate the smell epithelium in the uppermost nasal passage. As an example, smoking a pipe can be used, in which passive smoking which is located next to a pipe smoker has a very pleasant fragrance. However, the pipe smoker does not itself perceive this smell when inhaling tobacco. Some embodiments of inhalers according to the invention therefore not only release active substances, but can also manipulate the tongue and/or the smell center of the nose of a user.

Thus, in some embodiments, it can be provided that aromas and volatile constituents of aerosol generated during inhalation can pass via the throat and the canine into the nasal cavity of a user, where they can be registered by the smell cells. In order to improve the smoking enjoyment and the taste, it can be provided that the nose can be supplied with flavouring substances from the outside or intraorally. The smell can result directly from the active substance generated, or can emanate from special aromas which can be incorporated, for example, in the mouthpiece (FIG. 1. 1) and/or a mouthpiece material. In addition, the mouthpiece (FIG. 1. 1) can consist of carrier material which, in the case of moisture or lip contact, can transport flavouring substances and flavouring substances into the interior of the mouth via the saliva. The saliva taste can be perceived as a real taste by the receptors on the tongue.

The control of the inhalation phase can, in some embodiments, take over, carry out and/or initiate a flap preferably attached in the mouthpiece (FIG. 1. 1). The flap can respond to suction. The flap can activate one or more control elements, for example heating unit. The flap can activate the heating sleeve. The flap can thus be arranged in the mouthpiece (FIG. 1. 1) and/or be designed such that a part of the aerosol can escape from the mouthpiece (FIG. 1. 1) during inhalation through nozzles in the direction of the nose.

The evaporator can have a display and/or a screen. The display and/or the screen can be arranged on or on the housing (FIG. 1. 18) of the evaporator. The display and/or the screen can be a touch display and/or have the functions of a touchscreen. In some embodiments, a separate mobile phone app or the like can thus be superfluous. Alternatively, the evaporator can be or can be controlled with or by a mobile phone app and/or can be in data exchange. Alternatively or additionally, the evaporator can have a switch which can be arranged, for example, on or on the housing.

In some embodiments, a switch and/or a display can be arranged displaceably on the surface of the device, in some embodiments displaceably relative to one another. The switch and/or the display can have a magnetic carrier material. In some embodiments, the magnetic carrier material can be configured to adhere to a smooth metal surface and to be released therefrom. In some embodiments, a user can thus place his/her function keys there or in such a manner that a comfortable handling can result for him/her. The control of the display can be programmed and/or configured such that it is active only on a surface on which the user has the contact with the placed-on elements. The surface of the device can have electrically conductive tracks or use induction.

The active substance precursor carrier can consist of and/or comprise a zeolite tube, and/or the active substance can be encapsulated in a sphere which can be filled with active substance and/or active substance constituents. When the zeolite tube and/or the sphere is heated, active substance and/or aerosols can be released. The active substance vapor can pass through an outer wall barrier which, in this case, can function as a molecular sieve (FIG. 1. 2) which can be configured to allow only desired substances to pass through. The outer wall barrier can be a part and/or an outer wall of the zeolite tube and/or of the sphere. The zeolite tube and/or the sphere can have the outer wall barrier.

The active substance can be encapsulated in one or more capsules, for example spheres. The capsules can be connected separably to one another and/or can have different materials and/or shapes. The materials of the active substance precursor carrier and/or of the capsules can bind active substances, which arise, for example, during the decarboxylation of cannabis, and/or release them upon further activation. Therefore, in some embodiments, the evaporator can also be utilized to extract CBD/THC from the cannabis plant as active substance and to store it in a medium. The stored active substance can then be released again upon a later activation.

As a particular medium, for example, ground cannabis flower material can be provided in a mixture of flour, water, butter, baking powder, sugar, which in some embodiments can alternatively or additionally be rolled, for example can be rolled into a #3 baking form. The medium and/or the baking form can correspond to the dimensions of the active substance precursor carrier and/or can be baked at a temperature of 110°. This baking unit can be finished in the evaporator, e.g. at temperatures of 180° C.-200° C. At temperatures of 180° C. to 200° C., CBD and/or THC can be released, so that these and/or aerosols formed therewith, for example, can be inhaled. The baked baking unit can be consumed after the finished baking.

In some embodiments, the evaporator can have a coupling. The coupling can be configured to dock an additional device and/or an additional system and/or to connect it to the evaporator. For example, an additional device can be configured and/or provided to store active substances and/or aerosols generated in the evaporator. In some embodiments, an additional device can comprise or be a tank, for example a tank for gaseous substances for inhalation, a tank and/or a device for admixing oxygen, a tank for liquids, a wet filter or the like. The additional device can comprise a battery and/or an accumulator in order to provide, for example, electrical energy, in particular for the heating sleeve and/or the heating sleeve.

The heating chamber (FIG. 1. 7) can be coated in such a way that a microscopic and/or nanoscopic architecture of the surface can minimize an adhesion of dirt particles on the surface. Alternatively or additionally, an absorbent surface can be provided, which can comprise a superabsorber or consist of a superabsorber. In some embodiments, the absorbent surface can be arranged on or at the surface of the heating chamber, and/or can be a part of the surface of the heating chamber. The heating chamber and/or the surface of the heating chamber can comprise a or the absorbent surface. In some embodiments, alternatively or additionally, a superabsorber can be provided, which can be arranged, for example, in the heating chamber. A superabsorber or the superabsorber can be or have a negatively charged superabsorber. The superabsorber can absorb moisture. In some embodiments, the heating chamber (FIG. 1. 7) can be or become positively statically charged, so that moisture can precipitate on the absorber surface. Provision can be made for the absorber surface to be able to be replaced after the inhalation operation.

In some embodiments, the active substance precursor carrier can consist of ceramic or have ceramic, and/or be or have a ceramic baking mould. Provision can be made for the ceramic baking mould to be able to be filled with a mixture of cannabis material and sugar. At a temperature of 185° C., the sugar can caramelize. A temperature of 185° C. can be ideal to extract the active substances from the cannabis plant and/or to store them in the caramel. The result, and/or the ceramic baking mould, can be used as a mouthpiece (FIG. 1. 1) which can release taste and/or active substance.

An evaporator according to the invention can be suitable in particular for “heat not burn”, and/or can implement a “heat not burn” principle. The evaporator can be configured to roast a cigarette, for example a commercially available cigarette, at a temperature of below 300° C., without harmful substances being able to be released by burning in the process. As in the case of a normal smoking process, the cigarette can be heated in stages from the front to the rear, for example in stages of 3 mm. In some embodiments, up to 20 puffs of smoke can be possible. The evaporator can be configured such that burning of the cigarette during smoking pauses can be ruled out, and/or the heating can take place such that burning of the cigarette during smoking pauses can be ruled out. It can be provided that a ratio of active substance to air, e.g. a volume ratio of active substance to air, is not too high. An or the ratio of active substance to air can correspond to that of a cigarette, for example a commercially available cigarette. In some embodiments, the longer the puff, the more nicotine to air can be present and/or contained in one or the aerosol.

Two-phase smoking can be expedient in the case of cannabis cigarettes. In one or the first heating phase, for example, cannabis can be dried, and/or in one or the second heating phase, one or the active substance can be released. In some embodiments, heating of the active substance precursor can be carried out in portions and/or in portions. One or the heating sleeve (FIG. 4. 3) can enclose a tubular active substance carrier on all sides. During each inhalation operation, so much active substance can be produced that an optimum and/or prescribed ratio to the inhalation volume, for example a proportion of active substance in one or the inhalation volume or/or aerosol, can arise. Provision can be made for the heating unit to be able to displace itself on the active substance precursor carrier during each inhalation operation. The displacement can correspond to one or the smoking process of a cigarette, and/or take place during or after a smoking process. However, the evaporator according to the invention can be configured such that combustion takes place. A or the pollutant reduction in the heating of active substances and/or active substance precursor can be achieved by active substances being able to be produced even at a temperature of the heating unit or of the heating sleeve which can be, for example, 150° C. to a maximum of 200° C. in the case of cannabis. In some embodiments, active substances can, for example, already be extracted at a temperature of 150° C. to a maximum of 200° C. and/or active substance-containing aerosol can be formed, since the amount of active substance, and/or, for example, the required heat to be supplied, can be small. Combustion can be ruled out at such temperatures.

The heating and/or heating can be controllable. The heating and/or heating can be very precisely controllable.

One problem in known inhalers and evaporators is the pollutant burden resulting from the combustion of biological material. In some embodiments, however, the pollutant burden can be reduced, and/or avoided, since the active substance precursor can be located in a closed chamber, and/or the heating sleeve and/or the heating chamber, which can be heated from the outside. In some embodiments, the chamber can correspond to the heating sleeve, and/or be a part of the heating sleeve. The volatile material can and/or must diffuse through the outer walls of the active substance precursor carrier by an expansion or expansion on heating and associated change in volume. The outer walls of the chamber can consist of a material or have a material which can filter or bind harmful substances. The material can be or have foamed metal. The material can alternatively or additionally have the property of changing its pore size under the influence of a temperature or the temperature. In some embodiments, the material can be or have one or more of zeolites, calcium oxide, silicon oxide, aluminum oxide and smaller amounts of magnesium oxide, manganese oxide, iron oxide, phosphorus pentoxide and calcium sulfide. The material can be a filter or serve as a filter, and/or be a filter material. The filter material can be or have a mixture of clay, blast furnace slag and copper oxide, for example.

The evaporator can have a molecular sieve (FIG. 1. 2). The molecular sieve can be or have a filter. The evaporator can have a filter which is or has a or the molecular sieve. A molecular sieve can comprise or be, for example, natural and synthetic zeolites or other substances which have a high adsorption capacity for gases, vapors and solutes having specific molecular sizes. By a suitable choice of the molecular sieve (FIG. 1. 2), molecules of different sizes can be separated. It can advantageously be provided that only the amount of active substance which is in the chamber passes through the filtering process and/or the filter and/or the molecular sieve. In some embodiments, it can be provided that the complete inhalation volume is not filtered.

It can be particularly unpleasant when inhaling active substances if condensate is formed which at some point passes into the mouthpiece (FIG. 1. 1). The condensate residues can be a consequence of the high vapor concentration in the chamber, in particular in the vicinity of a or the evaporation surface or vapor outlet surface. The formation of condensate residues can be determined substantially by a diffusion in the direction of cooler boundary walls, and can take place in a flash-like manner. In one embodiment of the invention, it can then be provided that the condensate is bound. This can be achieved by the inner walls, for example of the heating chamber and/or of the heating sleeve, being able to be coated with a material. The material can have and/or bring about a lotus effect, for example. The heating chamber (FIG. 1. 7) can have different zones and/or regions in which a superabsorber (FIG. 1. 10) can be located. The superabsorber or superabsorbers can be exchangeable.

An interesting observation is that in all inhalers and evaporators, and especially in smoking, a pleasant taste and/or flavour is frequently missed, or at least would be desirable. This occurs especially in smokers. Thus, many cigarette manufacturers are forbidden to admix flavouring substances and flavouring substances into their cannabis products.

A generated aerosol can usually scarcely transport or impart a pleasant taste. Taste is usually activated on the tongue. Tests have shown that in a combination of inhalation, e.g. via the lungs and/or intraoral mucosa, smells, e.g. in the throat and nose, and tastes, e.g. by means of tongue, a very intense taste experience can be generated during inhalation. The taste can e.g. substantially take place via the nose. Only together with the smell can an aroma of an aerosol arise and/or be perceived. It can frequently be easier to transmit smell with a or the aerosol than to generate taste in the mouth and/or on the tongue. A generation of taste is frequently achieved only with the inclusion of saliva, which can activate the taste buds on the tongue.

Embodiments of the present invention can overcome these disadvantages. In some embodiments, it can be provided that taste and/or flavouring substances are released exclusively via the mouthpiece (FIG. 1. 1). This can be achieved by the mouthpiece (FIG. 1. 1) partially consisting of sugar and/or derivatives or derivatives thereof or being able to comprise those which are treated with taste and/or smell, and/or. active substance concentrates. The taste can be activated and controlled via the lips, and in some embodiments can then be or can be transmitted for example to the tongue. Provision can be made for taste sensations to be provoked and/or caused on the tongue, which can also be readily inhalable. A further embodiment can comprise a corresponding method, for example a method described above, and/or be set up to carry out a corresponding method. Commercially available cigarettes, cigarette sticks, in particular cigarettes with ingredients from the cannabis plant, so-called hemp cigarettes, can also be heated without burning.

In other words, in some embodiments, starting from an inhalation device of the type described at the outset, a cigarette substitute which is as complete as possible is to be created, specifically both with regard to the pharmacological and pharmacokinetic effects and with regard to the organoleptic effects of the vapor-air mixture or condensation aerosol generated. In some embodiments, the inhalation device is to be ergonomically handled and is to be configured as compact and space-saving as possible, so that use as a pocket evaporator is possible.

An evaporator can be or have an inhaler or an inhalation device, and/or serve as such and/or be or be referred to as such.

Accordingly, an evaporator can have a heating sleeve (FIG. 4. 3), which can enclose a cigarette, preferably on a length of 3-5 mm. As in a smoking process of a commercially available cigarette, during each puff on the cigarette, the heating sleeve can migrate along the cigarette, for example in the direction of a or the filter. In some embodiments, the evaporator can have a or the filter, which can be arranged in the heating chamber and/or on or at the mouthpiece. Normal or usual smoking of a cigarette can be simulated and/or simulated, but at a temperature of a maximum of 300° C. generated by the heating sleeve (FIG. 4. 3). Active substances can thus be released without burning the cigarette. The cigarette can be located and/or arranged, for example, in a cylindrical lattice cage. The cylindrical lattice cage can thus ensure that the cigarette can be removed from the evaporator without problems after smoking. It is also conceivable that the lattice cage can consist of individual zones or can have individual zones, which can be heated. In this case, the heating sleeve (FIG. 4. 3) can serve as energy supplier, which can supply one or more of the respective zones with energy.

A substantial part of the heat supplied in the course of the evaporation can remain in the heating sleeve (FIG. 4. 3). It can thus be prevented that the thermal or inner energy in the heating sleeve (FIG. 4. 3) can heat the heating space too much, e.g. in the case of inhalations following several times in succession. However, as the temperature increases, the saturation vapor pressure on the walls of the heating sleeve (FIG. 4. 3), which can serve as a filter unit, can also increase, which can lead to the pressure which the generated aerosol exerts on the filter elements and/or walls in the gas phase being relatively high. However, this cannot be harmful to the filter function, e.g. of the walls and/or filter unit.

The condensate residues can be accommodated, discharged and/or stored at the location of their formation, e.g. in the chamber, for example in the heating chamber and/or the heating sleeve, by one or more superabsorbers (FIG. 1. 10). It can be provided that, in some embodiments, neither the air flowing into the chamber nor the vapor-air mixture or condensation aerosol formed flows or can flow through the pores of the superabsorber (FIG. 1. 10). In some embodiments, the pores also cannot serve to store the active substances to be evaporated or to convey them to the evaporator. In some embodiments, the pores of the superabsorber (FIG. 1. 10) can serve, in at least one embodiment, exclusively to bind condensate residues by capillary action.

In the case of an air volume, e.g. a usual air volume, and/or an air volume of 0.5 to 1.0 L, a small amount of cooling liquid, e.g. of 20 to 80 mL, is sufficient to bring about sufficient cooling. The cooling liquid can preferably consist of or have water and/or other coolants. Different coolants can be distinguished, inter alia, by a germicidal and solubilizing action. In addition, preservatives such as carboxylic acids, e.g. sorbic acid or benzoic acid, can be contained in the cooling liquid. As a result of the addition of carboxylic acids, the cooling liquid can also be acidified to such an extent that it is able to accommodate and/or bind harmful substance released from the vapor-air mixture or condensation aerosol.

The evaporator and/or the mouthpiece (FIG. 1. 1) can have a liquid barrier in the form of a superabsorber (FIG. 1. 10). The superabsorber can be or have a zeolite, for example. Zeolites are important materials for numerous industrial and consumer products and have been intensively utilized for several decades. Zeolites are very porous, because their framework-like structure is permeated by countless nano-to micrometer-sized pores and channels. They resemble in their structure a sponge with many (micro-or nano-) holes, but are not flexible. Because of such nanoscale pores, they belong to the very few material classes which are considered to be nanomaterials not only because they form nanoscale particles, but also because they can have nanopores. They are of interest primarily as catalysts in chemical processes, since liquids can move freely through the pores and chemical reactions can take place on their walls. They can also be utilized for filtrations: because of the nanopores, even very small substances can be separated, which is then called nanofiltration.

The open-pored pore body or a or semipermeable membrane of a or the filter and/or of a or the superabsorber can be permeable to the vapor-air mixture or active substance, but hydrophobic material properties thereof can prevent harmful substances from passing into the chamber and/or escaping via the mouthpiece (FIG. 1. 1), in some embodiments for example independently of the spatial position and/or arrangement in the evaporator. Suitable hydrophobic material is in principle any material which is hydrophobic by nature and material which is hydrophobicized by suitable methods. PTFE (polytetrafluoroethylene) may be mentioned as a typical example of a material which is hydrophobic by nature.

However, the invention is not restricted to the described active substances and/or material, and/or chemically inert material. In a preferred embodiment, reconstituted cannabis, expanded cannabis or mixtures thereof can be provided. The cannabis can be present for example as cut cannabis or ground cannabis. Examples of suitable flavouring substances are cannabis extract, cannabis flavouring oils, menthol, coffee extract, cannabis smoke condensate or a volatile aromatic fraction of a cannabis smoke condensate. Of course, the invention is not restricted to this selection. The cannabis filling can be formed from cut cannabis, fine-cut cannabis, stuffing cannabis, from a cigar-like cannabis wrap or from comparable or similar cannabis forms. Suitable cannabis are in particular dried fermented cannabis, reconstituted cannabis, expanded cannabis or mixtures thereof. The cannabis can additionally be sauced, spiced, flavoured or/and perfumed by adding to it aromatic additives such as, for example, cannabis extract, cannabis flavouring oils, menthol, coffee extract, cannabis smoke condensate or a volatile aromatic fraction of a cannabis smoke condensate. The active substance precursors, for example, cannabis flower material, usually produce no or little qualm on heating. This can be changed by admixing different substances, for example propylene glycol, within the scope of the (German) Tobacco Products Act.

Propylene glycol has proven successful for transporting the active substances. The boiling point can be approximately 188° C., which can be equivalent to the release of active substances in cannabis flowers. The smoke can thus be interpreted as a visible indicator of the temperature.

Section 13 of the German Tobacco Products Act stipulates that no ingredient (other than nicotine) which could pose a risk to human health may be contained in liquid.

Propylene glycol (PG) E1520 PG (1.2 propanetriol) is approved as a food additive with the designation E1520. PG can also be found, inter alia, in inhalers, fog systems in discotheques, creams, etc. PG is a clear, colorless, almost odorless and highly hygroscopic liquid. Glycerol (VG) E422VG bears the designation E 422 as an approved food additive. VG is present as a fatty acid ester in all natural fats and oils and plays a central role as an intermediate in various metabolic processes.

The active substance aerosol particles produced by condensation can generally have a mass median aerodynamic diameter (MMAD) of less than 2 μm. They can thus also reach the alveoli, where the active substance can pass into the blood circulation. This transition can take place quickly and/or in a flash-like manner. Already about 7-10 seconds after inhalation, the active substance can reach its target organ, namely the central nervous system, in a concentrated concentration.

In the case of use of heating conductor alloys instead of stainless steel, a heating element resistance, for example of the heating sleeve and/or heating sleeve, can be significantly increased. For example, in the case of use of DIN material number 2.4872 (NiCr20AlSi) in comparison with AlSI 304/AlSI 316, this can result by a factor of 1.8 and in the case of use of DIN material number 1.4765 (CrAl255) even by a factor of 2.0.

The temperature in the heating sleeve (FIG. 4. 3) can be controlled as desired by an electrical circuit or the electrical circuit. Tests have shown that intermittent heating can considerably reduce the risk of burning of the active substance precursor. Use of bimetal in use with memory spring elements can be provided. Bimetal switches have proven successful in many heating devices, for example in the temperature regulation of irons and boilers, for controlling coffee machines, toasters and kettles. In a or the temperature switch, for example, a or the bimetal strip—in conjunction with a memory metal or magnetic adhesion—can open or close a contact depending on the temperature. The contact can for example switch on or off a or the heating sleeve (FIG. 3. 8) in the heating sleeve (FIG. 4. 3). For temperature control of the heating sleeve (FIG. 3. 8), one end of the bimetal strip can be fixed to the heating sleeve (FIG. 3. 8) and the other end can be provided with an electrical contact. The bimetal strips can either themselves have or have current flowing through them, or can carry a heating winding.

If, for example, a bimetal strip is placed and/or arranged in a or the supply line of the heating sleeve (FIG. 3. 8) of the heating sleeve (FIG. 4. 3), the heating sleeve (FIG. 3. 8) can begin to glow after a short heating time. As a result of the heating, the current flow from the bimetal to the heating sleeve (FIG. 3. 8) can be interrupted. On cooling, the circuit can close again. Correspondingly, the heating of the heating sleeve (FIG. 4. 3) can also take place.

In the case of a metallic heating sleeve (FIG. 3. 8), a single lithium-polymer or lithium-ion cell with an open circuit or rated voltage of approximately 3.7 V can be sufficient. A simple control strategy can consist of or comprise the steps of dividing the duration of the energy supply into a plurality of heating cycles, for example into a heating period and a subsequent evaporation period. In an or the intermittent, train-synchronous operation of the evaporator, the duration of the energy supply can be oriented to and/or correspond to the duration of a puff. In some cases, an average puff duration of approximately 2.1 sec (+/−0.4 sec) can be assumed. The same value can also apply approximately to cigarettes. If it is taken into account that, even after the energy supply has been switched off, a post-evaporation can take place to a certain degree on account of the heat still stored in the evaporator, it can appear expedient to select the duration of the energy supply to be somewhat shorter, e.g. a value in the range of 1.5 to 1.8 sec, e.g. for, during or at the first of the previously mentioned two periods. During the heating period, the heating sleeve (FIG. 4. 3) together with a part of the active substances stored in the active substance carrier can be heated by the heating element. It can be provided that the evaporation of the active substances can only begin when the temperature of the heating sleeve (FIG. 4. 3) has approximately reached the boiling range of the low-boiling fractions of the active substances. The heating period should therefore be as short as possible. In this respect, in some embodiments, it can be provided that the rechargeable battery voltage is conserved in this period and passed on to the heating element via a capacitor with a degree of modulation or duty cycle of 100%. The duration of the heating period can depend on the temperature required for the release of active substances, the specifications of the evaporator and/or on the amount and/or composition of the active substances. The heating period can be <0.5 sec. In the subsequent second period of the evaporation period, the degree of modulation can be substantially reduced, and one or the actual evaporation of the active substances can take place.

The supplied energy can be used in the second period primarily for evaporation of the active substances and secondarily for covering energy losses. By a corresponding choice of the degree of modulation, the evaporation capacity and thus also the amount of active substances evaporated per puff or inhalation can be controlled within certain limits. An upper limit can be set by use of bimetals for temperature control. Local drying out and overheating of the active substance can thus be ruled out. By a reduction or throttling of the degree of modulation, on the other hand, a thermal decomposition of a or the material, and/or of the active substance, can be counteracted.

The control strategy can be expanded and refined as desired: for example, it can be expedient to also take into account the state of the battery in the control strategy, since the battery voltage can drop significantly with increasing discharge and increasing age of the battery, especially under load. This effect can be countered by a shortening of the degree of modulation. In order to be able to carry out this correction also in the heating period, it is expedient to modulate the battery voltage of a new, charged battery not to 100% as previously proposed, but rather, for example, only to 80%, so that there can still be sufficient room for an adaptation. A condenser can be used to protect the batteries. A long service life of the batteries can be necessary if or because as many inhalations as possible are to be able to be carried out with the evaporator.

If the hollow cylinder contains or comprises, for example, 1-2 g of effectively utilizable active substance, and/or provides such, and/or the material contains or comprises active substance in a concentration of typically 1-5% by volume, then up to 75 trains or inhalations can be carried out with an evaporator according to the invention. For example, 100 μg of active substance can be evaporated per inhalation. 75 trains can correspond to approximately 8 cigarettes. If only 50 μg of active substance are evaporated per train, then the range can increase to 150 trains or inhalations, which value can correspond to approximately 1 pack of cigarettes.

Although the evaporator can be embodied extensively in the exemplary embodiments, the invention is of course not restricted to these embodiments. The evaporator can rather have any desired shape as long as it has a cylindrical active substance precursor or active substance precursor carrier arranged in the heating chamber (FIG. 1. 7) and a heating sleeve (FIG. 1. 8) arranged in the heating chamber (FIG. 1. 7). Heating sleeve (FIG. 4. 3). The heating sleeve can have vapor exit surfaces composed of zeolites, from which generated vapor can pass over and/or enter the heating chamber (FIG. 1. 7). Finally, the evaporator could also be heated with induction heat, radiant heat or microwaves instead of with ohmic heat. Non-electrical heat sources can also be utilized for the evaporation of the active substance. The direct utilization of chemical heat of reaction may be mentioned as an example. Finally, it should also be mentioned that the transport of the active substances can be considerably improved by the additional admixture of alcohol.

The features of the invention disclosed in the above description, in the drawings and in the claims can be essential both individually and in any desired combination for the realization of the invention.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1-48. (canceled)

49. An evaporator for extracting and filtering active substances from biological materials by heating, wherein an active substance precursor in the form of a hollow cylinder, the outer walls of which are permeable to aerosols, is arranged in a heating chamber, wherein the active substance precursor is surrounded by a heating sleeve which is seated displaceably on the active substance precursor, wherein the length of the active substance precursor is a multiple of the width of the heating sleeve, preferably the width of the heating sleeve is approximately 5% to 25% of the length of the active substance precursor, so that a plurality, preferably approximately 4 to 15, partial regions of the active substance precursor can be heated individually, so that active substance provided by the active substance precursor can be smoked at least in regions and/or an aerosol which comprises active substance provided in regions by the active substance precursor can be generated.

50. The evaporator according to claim 49, which has a mouthpiece which is configured to discharge and/or release an aerosol and/or air from the evaporator.

51. The evaporator according to claim 49, in which the heating chamber is double-walled with an inner wall and an outer wall, wherein a cavity is formed between the inner wall and the outer wall, wherein the heating chamber is preferably a double-walled cylinder, the inner diameter of which is preferably at least twice a diameter of the active substance precursor.

52. The evaporator according to claim 51, in which a vacuum exists in the cavity formed by the inner wall and the outer wall of the heating chamber or an active substance and/or coolant is located in the cavity.

53. The evaporator according to claim 49, in which one or the inner wall of the heating chamber has a nanocoating.

54. The evaporator according to claim 50, which has a filter on a region of the heating chamber facing the mouthpiece, wherein the filter preferably comprises one or more of activated carbon, sponge and/or wet filter, statically charged metal wool, meltblown nonwoven or combinations thereof.

55. The evaporator according to claim 50, which has a superabsorber on a region facing the mouthpiece, which superabsorber is configured to collect condensate.

56. The evaporator according to claim 49, which has a line which is configured to conduct outside air from an air inlet opening of the evaporator into the mouthpiece of the evaporator and/or into the heating chamber, wherein the line is preferably arranged in the heating chamber and/or preferably in one or the cavity formed by the inner wall and the outer wall of the heating chamber.

57. The evaporator according to claim 49, in which an outer wall of the evaporator is coated with thermochromatic lacquer which is configured to change its colour as a function of temperature.

58. The evaporator according to claim 49, wherein the evaporator has a housing which consists of a magnetic wire grid or has a magnetic wire grid, wherein the magnetic wire grid is coated extensively with plastic which, when current flows through wires of the wire grid, insulates the current-conducting wires from one another.

59. The evaporator according to claim 58, which has at least one switching element arranged on or at a surface of the housing, wherein the switching element is preferably inductively coupled to at least one wire and/or the magnetic wire grid.

60. The evaporator according to claim 49, in which the active substance precursor forms a receptacle, preferably a cylindrical receptacle, and/or is dimensioned such that it has the at least one length of 8 cm, preferably at least 8.4 cm, and a diameter of at least 0.6 cm, preferably at least 0.8 cm, wherein the active substance precursor is preferably dimensioned such that it completely accommodates a cigarette.

61. The evaporator according to claim 49, in which the active substance precursor and/or the receptacle has a cylindrical wire basket, which is preferably divided into at least five zones, wherein the zones preferably have no electrical contact with one another and/or are electrically insulated from one another.

62. The evaporator according to claim 61, wherein the heating sleeve is movable and at least partially encloses the active substance precursor, and wherein the heating sleeve has sliding contacts, which are set up to supply the individual zones of the wire basket with current and/or to heat them, preferably in order to heat active substances accommodated in a zone and/or in order preferably to form an aerosol.

63. The evaporator according to claim 49, which has a sliding element, which is arranged outside the heating chamber and which is coupled to the heating sleeve, so that a movement of the sliding element can be transferred to the heating sleeve and/or vice versa.

64. The evaporator according to claim 63, in which the sliding element is elastically connected to the heating sleeve, wherein the elastic connection is or preferably has a silicone tube and/or electrically conductive material.

65. The evaporator according to claim 63, in which the sliding element has an active substance chamber, in which active substance can be received, and the silicone tube is preferably configured to transport active substance from the active substance chamber into the heating chamber and/or the heating sleeve.

66. The evaporator according to claim 65, in which the active substance chamber can be reduced in size by pressure with the aid of a membrane, so that active substance can be fed into the silicone tube, the heating sleeve and/or the heating chamber.

67. The evaporator according to claim 65, in which the evaporator has a deflection roller which is configured to transport active substance into the heating chamber and/or the heating sleeve, wherein the deflection roller preferably has convex bulges and/or is preferably configured as a peristaltic pump.

68. The evaporator according to claim 49, in which the heating sleeve has a housing chamber wall which has a filter material through which air and/or aerosol formed in the heating sleeve, and/or aerosol formed in the region of the active substance carrier enclosed by the heating sleeve, can be diffused.

69. The evaporator according to claim 68, wherein the housing chamber wall and/or the filter material has pores which are configured to change their size as a function of temperature.

70. The evaporator according to claim 49, wherein one or the housing of the heating sleeve consists of a material or has a material which filters or binds harmful substances, wherein the material preferably comprises one or more zeolites, preferably inactivated zeolites with a grain size of 0.1 to 2 mm, marble, activated carbon, clay, ash and/or a mixture thereof.

71. The evaporator according to claim 49, in which the heating sleeve has a seal, preferably a sealing lip, which seals the heating sleeve and/or which is configured such that, when the heating sleeve is moved, it compresses and/or reduces a region of the heating chamber, preferably a region of the heating chamber arranged between seal and mouthpiece.

72. The evaporator according to claim 49, in which the heating sleeve has a or the seal, preferably a or the sealing lip, which is configured such that, when the heating sleeve is moved, it conveys air, active substance and/or aerosol, preferably through a or the porous housing of the heating sleeve.

73. The evaporator according to claim 49, wherein the heating sleeve has a heating sleeve.

74. The evaporator according to claim 73, in which the heating sleeve has a memory alloy or consists of a memory alloy, wherein the heating sleeve and/or the memory alloy is configured to expand on heating and to contract on cooling.

75. The evaporator according to claim 73, wherein the heating sleeve has a front sealing disk and a rear sealing disk, between which the heating sleeve is arranged and which are connected to the heating sleeve, wherein the front and/or the rear sealing disk is configured to change its diameter.

76. The evaporator according to claim 75, in which the front sealing disk and/or the rear sealing disk each have a first part and a second part, wherein preferably the first part and the second part are substantially semi-circular and/or semi-annular, wherein the respective first part is connected to the respective second part via a spring, so that the diameter of the respective sealing disk is changeable, wherein preferably the spring comprises a memory metal or consists of such a metal, so that preferably the spring expands or contracts depending on temperature.

77. The evaporator according to claim 75, in which the front sealing disk and/or the rear sealing disk is bendable, preferably bendable in and/or by an angle of up to 130°, so that the diameter of the respective sealing disk is changeable by bending between an unbent position and a bent position, wherein preferably the front sealing disk and the rear sealing disk is configured to be bendable depending on temperature.

78. The evaporator according to claim 77, in which the front sealing disk and/or the rear sealing disk is clamped to and/or contacts a or the inner wall of the heating chamber when the respective sealing disk is unbent, and the respective sealing disk is displaceable and/or movable in the heating chamber when the respective sealing disk is bent.

79. The evaporator according to claim 49, which is configured to generate aerosol in the heating chamber by mixing active substance with air, preferably outside air.

80. The evaporator according to claim 50, which is configured to generate aerosol by mixing active substance with air, preferably outside air, in the mouthpiece.

81. The evaporator according to claim 50, in which the mouthpiece comprises a flavouring substance and/or fragrance, wherein preferably the flavouring substance and/or fragrance is encapsulated and/or preferably the mouthpiece has a porous material, through which flavouring substance and/or fragrance can pass.

82. The evaporator according to claim 81, in which the flavouring substance and/or fragrance is encapsulated in a cellulose polymer, preferably a cellulose polymer of wood pulp.

83. The evaporator according to claim 81, in which the mouthpiece has a reservoir for the flavouring substance and/or fragrance and/or in which the mouthpiece is configured to add and/or admix the flavouring substance and/or fragrance with aerosol and/or air conveyed through the mouthpiece.

84. The evaporator according to claim 50, which has a flap arranged in the mouthpiece, which is configured to respond to suction, such that, when the flap responds to suction through the mouthpiece, a control element and/or the heating sleeve is activated and/or a or the aerosol is formed in the heating chamber and/or in the mouthpiece.

85. The evaporator according to claim 50, in which the mouthpiece has a nozzle through which aerosol and/or air can be discharged from the mouthpiece.

86. The evaporator according to claim 49, in which the active substance precursor is configured to accommodate active substance, and/or in which the active substance precursor has a receptacle and active substance accommodated in the receptacle.

87. The evaporator according to claim 86, in which different active substances are respectively accommodated in the receptacle in at least two different regions of the active substance precursor.

88. The evaporator according to claim 49, in which the active substance precursor has a filter material and/or consists of a filter material which is configured to bind harmful substances arising during a heating process, wherein preferably the filter material comprises zeolite.

89. The evaporator according to claim 49, in which the active substance is encapsulated, preferably encapsulated in spheres, wherein preferably a shell of the respective capsules is configured to bind and/or filter harmful substances.

90. The evaporator according to claim 49, in which the active substance is solid, liquid and/or gaseous and/or in which the active substance precursor is configured to accommodate active substance in the solid, liquid and/or gaseous state.

91. The evaporator according to claim 49, in which the active substance precursor is configured to bind active substances, which preferably arise during the decarboxylation of cannabis, and release them upon further activation, preferably upon heating.

92. The evaporator according to claim 49, in which the active substance comprises ground cannabis flowers, and/or a mixture of ground cannabis flowers and flour, water, butter, baking powder, and/or sugar.

93. The evaporator according to claim 49, wherein the evaporator has a coupling which is configured to dock an additional device on the evaporator and/or to connect it to the evaporator, wherein preferably the additional device is or has a tank, preferably a tank for storing or providing gaseous substances, a vacuum pump, and/or a wet filter.

94. The evaporator according to claim 50, which has a liquid reservoir which is arranged on or in the mouthpiece and which is configured as a wet filter, wherein preferably the liquid reservoir is arranged in such a way that generated aerosol and/or air can be conducted through the liquid reservoir.

95. The evaporator according to claim 94, in which the liquid reservoir is configured to clean generated aerosol and/or to treat it with taste and/or smell.

96. The evaporator according to claim 49, in which the heating sleeve is configured to heat the active substance carrier to a temperature of up to 300° C., preferably to up to 180° C.
